(19) United States
(12) Patent Application Publication
(10) Pub. No.: US 2003/0157584 A1
(43) Pub. Date: Aug. 21, 2003

(54) Liquid for assaying alpha-amylase activity

(76) Inventors: Kazuya Kawasaki, Saitama (JP); Osamu Seshimoto, Saitama (JP)

Correspondence Address: BIRCH STEWART KOLASCH & BIRCH, PO BOX 747, FALLS CHURCH, VA, 22040-0747, US

(21) Appl. No.: 10/345,135

(22) Filed: Jan. 16, 2003

(30) Foreign Application Priority Data
Jan. 18, 2002 (JP) 009578/2002

(51) Int. Cl. C12Q 1/40; G01N 33/573

(52) U.S. Cl. 435/22; 435/7.4

(57) ABSTRACT

An object of the present invention is to provide a liquid for assaying α-amylase activity that allows for an assay of α-amylase having an acidic optimum pH with high sensitivity. The present invention provides a liquid for assaying α-amylase activity which comprises 5 mM to 50 mM NaCl and 0.09 mM to 0.9 mM CaCl₂.

### TECHNICAL FIELD

The present invention relates to a liquid for assaying α-amylase activity and a method for assaying α-amylase activity using the same. More particularly, the present invention relates to a liquid for assaying α-amylase activity which comprises a predetermined concentration of salts, and a method for assaying α-amylase activity using the same. The liquid for assaying α-amylase activity and method for assaying α-amylase activity according to the present invention are useful for the quality control of agricultural crops or the like.

### BACKGROUND ART

Amylases in wheat include α- and β-amylases, which hydrolyze starch in the wheat as substrate to produce dextrin and maltose. In bread-making using wheat flour obtained by milling wheat, the quality of wheat flour is evaluated based on the visco-elasticity of the bread dough obtained therefrom, but this visco-elasticity may greatly depend on the amylase activity of the wheat flour.

Generally, β-amylase may be expressed in wheat while α-amylase may be expressed in nearly-germinating wheat. When bread is made by using wheat flour obtained by milling wheat containing some nearly-germinating wheat, the viscosity of the dough is reduced, indicating that the wheat flour has a lower quality. Therefore, it is important to know the amylase activity of wheat at the time of milling the wheat.

Generally, α-amylase activity is assayed by using a p-nitrophenol (PNP)-labeled oligosaccharide substrate and the coupling enzyme α-glucosidase based on the rate of PNP production. However, the e (molar absorption coefficient) of PNP is pH-dependent, that is, the e may exhibit its maximum value at pH9 or higher and decrease as the pH level goes down. Actually, given that the e value is 100 at pH8, it may be approximately 35 (i.e., reduced by a factor of about three) at pH6.5. Thus, in the assay for α-amylase activity when the optimum pH is acidic, there is a problem of lower sensitivity.

In particularly, α-amylases derived from crops have an optimum pH of 5 to 6, and thus it is important to improve the sensitivity in the activity assay. In order to improve the sensitivity in the activity assay, an oligosaccharide substrate labeled with chloronitrophenol (CNP), of which e is less pH-dependent, has been developed, although an activity assay of α-amylases derived from crops using CNP-substrate cannot attain as high sensitivity as one using PNP substrate. Since e (CNP) is higher than e (PNP) at pH5-6, it seems that α-amylases derived from crops have less activity on CNP-labeled oligosaccharide substrate than on PNP-labeled oligosaccharide substrate.

Currently, reaction is performed by using PNP-labeled oligosaccharide substrate at its optimum pH (pH5.4) and is quenched with an alkali solution, and the e of PNP is increased. However, this procedure is an end-point method, that is, blank test (i.e., without substrate) is performed separately (substrate blank), and the substantial rate of PNP production is determined from the difference between the results with and without the substrate. Since two assays must be performed to determine one α-amylase activity in this procedure, there is a problem that the procedure is complicated .

### DISCLOSURE OF THE INVENTION

It is an object of the present invention to solve the aforementioned problems of the background art. One object of the present invention is to provide a liquid for assaying α-amylase activity that allows for an assay of α-amylase having an acidic optimum pH with high sensitivity. Another object of the present invention is to provide a method for assaying α-amylase activity that allows for an assay of α-amylase having an acidic optimum pH with high sensitivity.

As a result of intensive studies, the present inventors have found that the activity of α-amylase of which optimum pH is acidic can be determined with a high sensitivity by using a liquid for assaying α-amylase activity which comprises a certain concentration of each of NaCl and CaCl₂. The present invention was completed based on these findings.

Thus, the present invention provides a liquid for assaying α-amylase activity which comprises 5 mM to 50 mM NaCl and 0.09 mM to 0.9 mM CaCl₂.

Preferably, the liquid for assaying α-amylase activity according to the present invention comprises 8.6 mM to 42.8 mM NaCl and 0.18 mM to 0.90 mM CaCl₂.

Preferably, the α-amylase is derived from crops, and particularly preferably, the α-amylase is derived from cereal.

According to another aspect of the present invention, there is provided a method for assaying α-amylase activity wherein the liquid for assaying α-amylase activity according to the present invention is used.

Preferably, the method for assaying α-amylase activity according to the present invention comprises steps of adding a liquid for assaying α-amylase activity according to the present invention which contains α-amylase to an analytical element which contains a p-nitrophenol-labeled oligosaccharide substrate and α-glucosidase, and determining the rate of p-nitrophenol production.

### EMBODIMENT OF THE INVENTION

Hereinafter, embodiments and procedures of the present invention will be described in detail.

The liquid for assaying α-amylase activity of the present invention comprises 5 mM to 50 mM NaCl and 0.09 mM to 0.9 mM CaCl₂. In the case of assaying α-amylase activity using the liquid for assaying α-amylase activity of the present invention, a liquid for assaying α-amylase activity is firstly prepared which comprises α-amylase, 5 mM to 50 mM NaCl and 0.09 mM to 0.9 mM CaCl₂. Then, the liquid for assaying α-amylase activity is added to an analytical element which comprises p-nitrophenol (PNP)-labeled oligosaccharide substrate and the coupling enzyme α-glucosidase, and the production rate of the generating PNP is determined, thereby determining α-amylase activity.

According to the present invention, it was found that, by adjusting the concentration range of NaCl at 5 mM to 50 mM and the concentration range of CaCl₂ at 0.09 mM to 0.9 mM, the activity of α-amylase having an acidic optimum pH can be determined with high sensitivity, while maintaining the stability of α-amylase. When the concentration of NaCl is less than 5 mM or the concentration of CaCl₂ is less than 0.09 mM, then the stability of α-amylase at a relatively higher temperature (e.g., 45° C.) may be reduced, which is not preferable. On the other hand, when the concentration of NaCl is more than 50 mM or the concentration of CaCl₂ is more than 0.9 mM, then the sensitivity of the assay of α-amylase activity may be reduced, which is not preferable.

Particularly preferable is the use of a liquid for assaying α-amylase activity which comprises 8.6 mM to 42.8 mM NaCl and 0.18 mM to 0.90 mM CaCl₂.

The liquid for assaying α-amylase activity of the present invention is particularly suitable for assaying α-amylase having an acidic optimum pH. Such α-amylases include, for example, those derived from crops (e.g., α-amylases derived from cereal such as wheat or barley).

According to the present invention, a method for assaying α-amylase activity is provided which involves use of the liquid for assaying α-amylase activity. More particularly, the liquid for assaying α-amylase activity of the present invention which comprises α-amylase may be added to an analytical element which contains p-nitrophenol-labeled oligosaccharide substrate and α-glucosidase, and the rate of p-nitrophenol production may be then determined, whereby α-amylase activity can be determined.

The analytical elements used in the above-described assay may be a dry analytical element. Examples of dry analytical element which can be used in the present invention include those which comprise two layers on a support wherein one of the layers contains at least p-nitrophenol-labeled oligosaccharide substrate and the other layer contains α-glucosidase (hereinafter sometimes referred to as a “reagent layer”). Alternatively, p-nitrophenol-labeled oligosaccharide substrate and α-glucosidase may be contained in the same reagent layer. A water absorption layer may additionally be provided between the support and the reagent layer, if desired.

Any suitable light-non-transmitting (opaque), light-semi-transmitting (semi-transparent) or light-transmitting (transparent) support can be used in the present invention. Generally preferable are light-transmitting and water-impermeable supports. Preferable materials for light-transmitting and water impermeable support include polyethylene terephthalate and polystyrene. Preferably, an undercoating layer may be provided on the support or the support may be subjected to hydrophilization treatment in order to firmly fix the hydrophilic layer.

The reagent layer comprises p-nitrophenol-labeled oligosaccharide substrate and α-glucosidase. As described above, these two components may be contained in the same layer or separately in different layers. To ensure water-permeability of the reagent layer, it may be a porous layer consisting of porous medium or may be a layer consisting of hydrophilic polymer binder. Among these water-permeable layers, a continuous layer consisting of hydrophilic polymer binder may be preferable.

When a porous layer is used as the reagent layer, the porous medium may be either fibrous or non-fibrous. Examples of fibrous material include filter papers, non-woven fabrics, woven cloth (such as plain cloth), knitted webs (tricot fabrics), and glass fiber filters. Examples of non-fibrous material include membrane filters comprising cellulose acetate and the like as disclosed in, for example, JP Patent Publication (Unexamined Application) No. 49-53888, or a layer of a particulate structure containing interconnected voids and consisting of organic or inorganic microparticles as disclosed in, for example, JP Patent Publication (Unexamined Application) Nos. 49-53888, 55-90859 (corresponding to U.S. Pat. No. 4,258,001) and 58-70163 (corresponding to U.S. Pat. No. 4,486,537). Also preferable are laminated products of partially bonded multiple porous layers as disclosed in, for example, JP Patent Publication (Unexamined Application) Nos. 61-4959 (corresponding to EP0166365A), 62-116258, 62-138756 (corresponding to EP0226465A), 62-138757 (corresponding to EP 0226465A), and 62-138758 (corresponding to EP0226465A).

Alternatively, the porous layer may be a developing layer having a so-called metering function of developing a liquid over an area substantially in proportion to the volume of the liquid fed thereto. Preferable materials for the developing layer are woven and knitted fabrics. The woven fabrics or like may be subjected to the glow discharge treatment as described in JP Patent Publication (Unexamined Application) No. 57-66359. The developing layer may optionally contain a hydrophilic polymer or surfactant as described in JP Patent Publication (Unexamined Application) Nos. 60-222770 (corresponding to EP0162301A), 63-219397 (corresponding to DE3717913A), 63-112999 (corresponding to DE3717913A) and 62-182652 (corresponding to DE3717913A) in order to control the developing area or the developing rate.

For example, a porous membrane of paper, cloth or polymer may advantageously be immersed in or coated with a reagent according to the present invention, and then laminated on another water-permeable layer which has been provided on a support by, for example, the method described in JP Patent Publication (Unexamined Application) No. 55-164356.

The thickness of the reagent layer prepared as described above is not particularly limited, and is suitably about 1 μm to 50 μm, and preferably about 2 μm to 30 μm when it is provided as a coating layer. When the reagent layer is prepared by any other method such as laminating, the thickness may be a wider range of tens to hundreds μm.

When the reagent layer is composed of a water-permeable layer of hydrophilic polymer binder, examples of hydrophilic polymer which can be used include: gelatins and derivatives thereof (such as phthalated gelatin), cellulose derivatives (such as hydroxyethyl cellulose), agarose, sodium alginate, acrylamide copolymers, methacrylamide copolymers, copolymer of acrylamide or methacrylamide with any vinyl monomer, polyhydroxyethyl methacrylate, polyvinyl alcohol, polyvinylpyrrolidone, sodium polyacrylate, and copolymer of acrylic acid with any vinyl monomer.

A reagent layer composed of hydrophilic polymer binder can be prepared by applying an aqueous solution or aqueous dispersion solution containing a substrate, other reagent composition(s) and a hydrophilic polymer onto a support or another layer such as a detection layer and drying it according to any of the methods disclosed in JP Patent Publication (Examined Application) No. 53-21677 (corresponding to U.S. Pat. No. 3,992,158), JP Patent Publication (Unexamined Application) Nos. 55-164356 (corresponding to U.S. Pat. No. 4,292,272), 54-101398 (corresponding to U.S. Pat. No. 4,132,528), and 61-292063 (Chemical Abstracts, 106: 210567y).

The reagent layer containing a hydrophilic polymer as a binder may generally have a thickness of about 2 μm to about 50 μm, preferably about 4 μm to about 30 μm as a dry product, and the coated amount is about 2 g/m² to about 50 g/m², and preferably about 4 g/m² to about 30 g/m².

In addition to p-nitrophenol-labeled oligosaccharide substrate and α-glucosidase, the reagent layer may also contain any additive such as surfactant, pH buffer composition, fine powder, anti-oxidant and any other organic or inorganic additives, in order to improve various characteristics such as coating characteristics, diffusibility of the diffusible material, reactivity and storage stability. Examples of buffer which can be contained in the reagent layer include pH buffer system which is disclosed in “KAGAKU BINRAN, KISOHEN” edited by Japanese Chemical Society (MARUZEN, Tokyo, 1966), pp.1312-1320; R. M. C. Dawson et al., “Data for Biochemical Research” 2nd edition, (Oxford at the Clarendon Press, 1969), pp. 476-508; Biochemistry, pp. 5,467-477 (1966); Analytical Biochemistry, 104, pp. 300-310 (1980). Examples of pH buffer include those containing borate; those containing citric acid or citrate; those containing glycine; those containing Bicine; those containing HEPES; and Good's buffer such as those containing MES.

The dry analytical element which can be used in the present invention can be prepared according to the methods described in, for example, JP Patent Publication (Unexamined Application) Nos. 49-53888 (corresponding to U.S. Pat. No. 3,992,158), 51-40191 (corresponding to U.S. Pat. No. 4,042,335), 55-164356 (corresponding to U.S. Pat. No. 4,292,272), and 61-4959 (corresponding to EPC 0166365A).

It is preferred in terms of manufacture, packaging, transportation, storage and manipulation that the analytical element which can be used in the present invention is cut into pieces of square shape (from about 10×10 mm to about 30×30 mm) or any suitable shape (e.g., round shape) of equivalent size, and placed in a slide frame such as those described in JP Patent Publication (Examined Application) No. 57-28331 (corresponding to U.S. Pat. No. 4,169,751), JP Utility Model Publication (Unexamined Application)No. 56-142454 (corresponding to U.S. Pat. No. 4,387,990), JP Patent Publication (Unexamined Application) No. 57-63452, JP Utility Model Publication (Unexamined Application) No. 58-32350 and JP Patent Publication (PCT Translation) No. 58-501144 (corresponding to International Publication WO83/00391) to prepare a slide for chemical analysis. Depending on the purpose for use, the analytical element may be of a long tape type which may be placed in a cassette or cartridge for use, or of a small piece which can be mounted on or embedded into a card having an opening or can be used directly.

By using the method for assay according to the present invention, the activity of α-amylase which is a subject in a liquid sample can be determined. For example, about 2 μL to about 30 μL, preferably 4 μL to 15 μL of sample (the liquid for assaying α-amylase activity according to the present invention which contains α-amylase) may be spotted onto the reagent layer. The spotted analytical element may be incubated at a constant temperature of about 20° C. to about 45° C., preferably about 30° C. to about 40° C. for 1 to 10 minutes. By monitoring color development or change in the analytical element, α-amylase activity can be determined.

The quantitative assay can be performed with high accuracy by using the same conditions as to the amount of liquid sample to be spotted, the incubation time and the temperature.

The present invention will be described in more detail with reference to the following examples, but these examples illustrate the present invention and does not limit the scope of the present invention.

### EXAMPLES

### Example 1

### Preparation of Dry Analytical Element for Assay of α-amylase Activity

A gelatin-undercoated polyethylene terephthalate colorless transparent smooth film (180 μm thick) was coated with an aqueous solution having the following composition (pH6.5) in such a way that the coating layer of the dry product had a thickness of 14 μm, and was dried.

| | |
|---|---|
| Gelatin | 14.0 g/m² |
| HEPES | 0.7 g/m² |
| Surfactant | 0.5 g/m² |

Surfactant was polyoxy (2-hydroxy) propylene nonylphenylether (Surfactant 10G available from OLIN Corporation).

HEPES represents N-2-hydroxyethyl piperazine-N′-ethane sulfonic acid.

Next, the above-prepared film was moisturized by supplying water over its entire surface (at about 30 g/m²), a tricot fabric woven cloth obtained by 36-gages knitting polyethylene terephthalate spun yam of approximately 50-denier was superimposed and gently pressed on the film, and the obtained laminate was then dried.

An aqueous solution having the following composition (pH6.5) was applied to the cloth, which was then dried.

| | |
|---|---|
| Polyvinylpyrrolidone | 4.4 g/m² |
| HEPES | 6.4 g/m² |
| BG7-PNP | 2.7 g/m² |
| Surfactant | 1.7 g/m² |

BG7-PNP represents 4,6-ethylidene-4-nitrophenyl-α-D-maltoheptaoside

Further, an aqueous solution having the following composition (pH6.5) was applied to the cloth, which was then dried to prepare an integrated multi-layered analytical element.

| | |
|---|---|
| Polyvinylpyrrolidone | 3.9 g/m² |
| HEPES | 1.7 g/m² |
| α-glucosidase | 120.0 KU/m² |
| Surfactant | 2.0 g/m² |

The integrated multi-layered analytical element was cut into pieces (rectangular tip: 12 mm×13 mm), and the pieces were then placed on slide frames (disclosed in JP Patent Publication (Unexamined Application) No. 57-63452) to prepare dry analytical elements for assay of α-amylase activity.

### Example 2

### Relationship Between Salt Concentration and α-amylase Activity

<Preparation of Sample>

To a solution of 1200 U/L α-amylase (from Barley Malt) in 0.5% BSA (Bovine Serum Albumin) was added CaCl₂ at a concentration of 0, 0.06, 0.11, 0.23, 0.45, 0.90, 1.80, 3.60 or 7.21 mM. Similarly, NaCl was added at a concentration of 0, 1.1, 2.1, 4.3, 8.6, 17.1, 42.8, 85.6 or 171.1 mM.

<Assay>

10 μl of each of these samples were spotted on the dry analytical elements prepared in Example 1, and incubated at 37° C. Then, the difference between the reflection ODs detected at 2.5 minutes and 5.0 minutes was determined (ΔODt).

The relationship between the CaCl₂ concentration and the sensitivity is shown in Table 1, and the relationship between the NaCl concentration and the sensitivity is shown in Table 2. The right column in Table 1 shows relative ΔODt values at various CaCl₂ concentrations when the ΔODt at CaCl₂ concentration of 1.8 mM is regarded as 100. The right column in Table 2 shows relative ΔODt values at various NaCl concentrations when the ΔODt at NaCl concentration of 85.6 mM is regarded as 100.

TABLE 1

| CaCl₂ conc. | ΔODt | % |
|---|---|---|
| 0 | 0.0539 | 115 |
| 0.06 | 0.0548 | 117 |
| 0.11 | 0.0537 | 114 |
| 0.23 | 0.0524 | 112 |
| 0.45 | 0.0511 | 109 |
| 0.90 | 0.0494 | 105 |
| 1.80 | 0.0469 | 100 |
| 3.60 | 0.0439 | 94 |
| 7.21 | 0.0390 | 83 |

TABLE 2

| CaCl₂ conc. | ΔODt | % |
|---|---|---|
| 0 | 0.0540 | 137 |
| 1.1 | 0.0542 | 137 |
| 2.1 | 0.0539 | 136 |
| 4.3 | 0.0536 | 136 |
| 8.6 | 0.0518 | 131 |
| 17.1 | 0.0500 | 126 |
| 42.8 | 0.0439 | 111 |
| 85.6 | 0.0395 | 100 |
| 171.1 | 0.0303 | 77 |

Table 1 shows that higher sensitivity can be obtained by lowering the CaCl₂ concentration as compared with the conventional case when the CaCl₂ concentration is 1.8 mM.

Table 2 shows that higher sensitivity can be obtained by lowering the NaCl concentration as compared with the conventional case when the NaCl concentration is 85.6 mM.

### Example 3

### Relationship Between Salt Concentration and α-amylase Stability

<Preparation of Sample>

CaCl₂ and NaCl were added to a solution of 1200 U/L α-amylase (from Barley Malt) in 0.5%BSA (Bovine Serum Albumin) to the following final concentrations:

Solution A: CaCl₂=0 mM, NaCl=0 mM;

Solution B: CaCl₂=1.8 mM, NaCl=85.6 mM; and

Solution C: CaCl₂=0.18 mM, NaCl=8.6 mM.

<Assay>

Solutions A-C were stored at 25° C. and 45° C. for 4 hours, and the change in the sensitivity was monitored. Sensitivity was determined by spotting 10 μl each of the above-described samples to the dry analytical elements prepared in Example 1, incubating the samples at 37° C., and determining ΔODt as the difference between the reflection ΔODs detected at 2.5 minutes and 5.0 minutes

Results are shown in Table 3 below.

TABLE 3
Salt concentration and α-amylase stability
45° C. (ΔODt)

| Time (min.) | 0 | 30 | 60 | 120 | 240 |
|---|---|---|---|---|---|
| Solution A | 0.0571 | 0.0565 | 0.0561 | 0.0553 | 0.0529 |
| Solution B | 0.0382 | 0.0381 | 0.0381 | 0.0381 | 0.0379 |
| Solution C | 0.0531 | 0.0530 | 0.0530 | 0.0525 | 0.0523 |

45° C. (% relative ΔODt when the value at time 0 is regarded as 100)

| Time (min.) | 0 | 30 | 60 | 120 | 240 |
|---|---|---|---|---|---|
| Solution A | 100 | 99 | 98 | 97 | 93 |
| Solution B | 100 | 100 | 100 | 100 | 99 |
| Solution C | 100 | 100 | 100 | 99 | 99 |

25° C. (ΔODt)

| Time (min.) | 0 | 30 | 60 | 120 | 240 |
|---|---|---|---|---|---|
| Solution A | 0.0555 | 0.0559 | 0.0554 | 0.0552 | 0.0548 |
| Solution B | 0.0366 | 0.0361 | 0.0354 | 0.0361 | 0.0357 |
| Solution C | 0.0510 | 0.0515 | 0.0513 | 0.0513 | 0.0517 |

25° C. (% relative ΔODt when the value at time 0 is regarded as 100)

| Time (min.) | 0 | 30 | 60 | 120 | 240 |
|---|---|---|---|---|---|
| Solution A | 100 | 101 | 100 | 99 | 99 |
| Solution B | 100 | 99 | 97 | 98 | 98 |
| Solution C | 100 | 101 | 101 | 101 | 101 |

Table 3 shows that the amylase activity is not decreased and is stable at 45° C. in solutions B and C.

### EFFECT OF THE INVENTION

By using the liquid for assaying α-amylase activity according to the present invention, α-amylase activity having an acidic optimum pH can be assayed with high sensitivity, and the stability of α-amylase can be maintained.

What is claimed is:

1. A liquid for assaying α-amylase activity which comprises 5 mM to 50 mM NaCl and 0.09 mM to 0.9 mM CaCl₂.

2. The liquid for assaying α-amylase activity according to claim 1 which comprises 8.6 mM to 42.8 mM NaCl and 0.18 mM to 0.90 mM CaCl₂.

3. The liquid for assaying α-amylase activity according to claim 1 wherein the α-amylase is derived from crops.

4. The liquid for assaying α-amylase activity according to claim 3 wherein the α-amylase is derived from cereal.

5. A method for assaying α-amylase activity wherein a liquid for assaying α-amylase activity according to claim 1 is used.

6. The method for assaying α-amylase activity according to claim 5 which comprises steps of adding a liquid for assaying α-amylase activity according to claim 1 which contains α-amylase to an analytical element which contains a p-nitrophenol-labeled oligosaccharide substrate and α-glucosidase, and determining the rate of p-nitrophenol production.
